(19) United States
(12) Patent Application Publication
(10) Pub. No.: US 2016/0271011 A1
(43) Pub. Date: Sep. 22, 2016

(54) Electronic Cardiopulmonary Resuscitation and Bleeding Control

(71) Applicant: Jerry Hughes, Austin (TR)

(72) Inventor: Jerry Hughes, Austin (TR)

(21) Appl. No.: 15/072,298

(22) Filed: Mar. 16, 2016

Related U.S. Application Data
(60) Provisional application No. 62/133,967, filed on Mar. 16, 2015.
(60) Provisional application No. 62/141,367, filed on Apr. 1, 2015.

(51) Int. Cl. A61H 31/00; A61N 1/36; A61N 1/04

(52) U.S. Cl. CPC A61H 31/006; A61N 1/0456; A61N 1/0452; A61N 1/0492; A61N 1/36014

(57) ABSTRACT

Lack of blood flow during a heart attack is controlled by the use of a portable, small-footprint electronic device that is connected to the patient using wires and connections/pads sending an electric current/pulse to the heart that is adjusted for intensity and rhythm to increase blood flow. The devices electronic signal overrides the bodies electronic signal to control the heart for a regular heartbeat. Using an electronic device to send a current to connections/pads on the abdomen and limb pulse points to add compression and constrict blood vessels reduces blood flow to these areas to increase blood flow to the heart and brain during cardiac arrest. Bleeding is controlled by the use of an electronic device to send an adjustable electronic current/signal to compress an area of the body and constrict blood vessels to control blood flow for both external and internal injuries.

### CROSS-REFERENCE TO RELATED APPLICATIONS

This application claims benefit of U.S. Provisional Application No. 62/133,967, filed Mar. 15, 2015, and U.S. Provisional Application No. 62/141,367, filed Apr. 1, 2015 each of which is hereby incorporated by reference.

### BACKGROUND OF THE INVENTION

1. Field of the Invention

The invention generally relates to a medical procedure method using known parts but steps of method have not been previously known or used. The medical procedure uses electricity to control heartbeat and blood flow in the body to increase blood flow or reduce it. In particular increasing blood flow by overriding the bodies electrical signals to the heart with ours to compress and release the heart in a rhythm to increase blood flow. In addition sending electricity to an injured area to constrict the blood vessels to reduce blood flow. This may be used with cardiac arrest and areas of injury both external and internal.

2. Description of Related Art

CPR, Cardiopulmonary resuscitation, is the standard procedure for treating a heart attack when a person is not breathing or has no pulse. Doing CPR is physically exhausting and even trained paramedics trade off after 4 or 5 minutes. A partner of an elderly person would not have the stamina to do it for long enough to be effective. The response time for an ambulance can be 15 minutes to an hour depending on traffic or if it is a rural area. multiple people would be needed to keep up CPR that long. Continuing CPR in the back of an ambulance on the way to the hospital with one paramedic driving and one in the back with the patient would not work on a trip to the hospital taking over 10 minutes. I talked with a paramedic who works in a rural farming area who said it could be a 90 minute drive from a farm to the local hospital. The combination of CPR being physically exhausting and length of response time that it has to be continued for makes it a deadly combination for a patient.

CPR must be started immediately when the patient is not breathing or has no pulse. The small window of time, 4 to 6 minutes to get blood to the brain before it starts to die, under 10 minutes before heart tissue begins to die and the heart loses its spark and ability to be restarted using defibrillation. Time is heart muscle and brain tissue and how quickly resuscitation begins is vital. Most people who are bystanders have not been trained in CPR and are reluctant to do CPR on a stranger. A person who is having a heart attack cannot do CPR on themselves. Without CPR people die.

Quick response is critical. Defibrillation units are expensive and generally located in buildings. If the trip to get the device is 3 minutes that makes it a 6 minute round trip and the patients brain is already dying. Defibrillation does not start the heart, it stops it. Then the heart must be in good enough shape to restart itself.

There is a point where the heart cannot restart itself. Overweight people are more likely to have a heart attack and are harder to administer CPR to. CPR alone is unlikely to start the heart, it is a stopgap measure to supply some blood to the brain and heart to delay tissue death and increase odds of successful resuscitation without permanent brain damage. CPR delivers only about 20 to 30% of normal heart blood flow. Survival rate is low. CPR also causes rib fractures, heart contusions, and lacerations to other organs. The Lucas is a very large expensive device that is not very portable and used after paramedics arrive or in a hospital. The critical first 5 to 10 minutes is before help arrives. Survival requires a device that is more portable and able to be used by anyone quickly on the location of the heart attack. 60% of people get a wake up call with their first heart attack, 40% never wake up, they die. A quicker and better way than current CPR is needed.

Controlling the bleeding on an external wound generally is done by applying pressure to the area of the wound. Holding a hand on the area may work to apply pressure but is not effective if there is more than one wound or multiple patients. Excess bleeding can cause shock, loss of life, and make coagulation harder. A tourniquet is sometimes used but is generally used as a last resort. Life or limb is the choice because you the patient will lose one or the other. Turning off all blood flow is drastic and causes tissue damage to the area below the tourniquet. Bleeding with internal injuries can be even harder to control especially if they are not on a limb but in the main body or abdomen. Applying pressure to control bleeding in these areas is much harder to do. Closing a wound like a bullet hole or knife wound is hard because in goes into the body. Bruising both external and internal can also cause infection. One of the most effective ways of treating a burn is immediately running cold water over it but what if there is no cold water available? In surgery bleeding is controlled with cauterization, clamps and tying. These methods kill tissue. Suction is used to clear blood from the area but does not reduce blood loss. Stitches are used to seal an incision.

CPR and compression to control blood flow cannot be adjusted at a second location like a hospital or by paramedics en route to a patient or controlled directly by being connected to monitoring for the patients vital signs and other activity. CPR and compression cannot be set to an automated program to adjust to the patient, type of injury or amount of time from time of injury.

### BRIEF SUMMARY OF THE INVENTION

Electronic Cardiopulmonary Resuscitation uses an electronic device to compress and release the heart in a regular rhythm to keep blood pumping to the brain, requires no physical exertion and can be done for up to 11 hours with one battery. For long response and transport times especially in rural areas this makes a big difference. We use the patients own heart muscle to pump blood by overriding their bodies electrical signals with a stronger signal from our electronic unit that tells the heart to compress and release. The unit can be set to send a signal like a pacemaker to trigger a heartbeat or set to form a heartbeat by building current to build compression squeezing the heart and then releasing it.

Electronic Cardiopulmonary Resuscitation can be used immediately during the first critical 5 to 10 minutes or longer while waiting for the ambulance and during the transport to the hospital as well as in the hospital. A persons partner can do it no matter what their physical strength. In some situations a person could actually attach it and start it going after calling 911 and when they were sure it was needed. Larger devices like the Lucas are expensive and not very portable, generally being used in an ambulance or at a hospital. The electronic unit is inexpensive and smaller than a cell phone. It can easily be carried in a purse or car. No training is required to use it as it is simply attaching the wires from the electronic device to the pads and placing them on the person, then starting the automatic program on the unit. People would be a lot more likely to use this medical procedure than doing chest compressions. Response time has been studied as a major factor in survival rates for heart attack patients. Electronic Cardiopulmonary Resuscitation reduces the impact of long response times (15 minutes can be a long response time with a heart attack) and transport factors as well a physical exhaustion found with regular CPR. It keeps the heart in better condition for restarting after defibrillation, using electricity helps keep the spark in the heart alive. (There is a point where the heart won't restart with defibrillation.) The brain also gets a better chance for recovery. Less death and better quality of life for survivors. For overweight people you can increase the amount of current. ECPR is does not cause the damage to the body that CPR does. Pads can be attached to the abdomen and pressure points for the limbs to reduce blood flow to those areas and increase blood flow to the brain and heart using a different channel on the electronic device or a second device. Linking the device to a cell phone will allow a medical professional like a paramedic or hospital staff to adjust the electronic device remotely. A port for adding a pad to read vital signs can also be added to the device which would allow them to be sent to the hospital as well.

EBC (Electronic Bleeding Control) allows a person to control bleeding in multiple areas and can even be self administered. Since the electronic devices are small and inexpensive they can be used on multiple patients at one time which is important in war zones and triage situations. Sending a steady current from the electronic unit through the wires to the pads on the patient constricts the blood vessels in the area of the wound to reduce bleeding. Reducing bleeding helps in coagulation, and helps avoid shock as well as infection in the injured area. Applying pads and current to a pressure point for a limb will reduce the blood flow to that area. This can be used alone or in conjunction with pads on the wound area. A strap around a limb connected to the electrical device will send current into that area constricting the muscles and blood vessels like a tourniquet but with less damage. The strap can be set to allow control the blood flow with enough blood flowing through to help keep the limb healthy and less compression is needed if used with pads over the injured area helping save not only the patients life but their limb as well. Bleeding from internal injuries can be controlled by placing pads over the area with electronic current flowing through them to constrict them and blood flow following an injury. Just like flowing cold water over a burn makes a big difference if done quickly after the burn takes place, restricting blood flow quickly with an internal injury helps slow down the damage. For burns if no cold water is available using a large pad or pads can help slow down the damage like cold water by constricting the area and blood flow. A bullet or knife wound can be hard to close internally. Electronic Bleeding Control helps close the bullet wound or cut on the inside as well as the outside area. In surgery using EBC helps keep tissue healthy by reducing bleeding without having to cauterize or clamp off areas. It also reduces the need for suction making it easier to see the area being worked on and there is less blood loss. Ceramic scalpels or insulated instruments can be used and a combination of electrical current and instruments can be effective too. Compressing the tissue together using EBC can help where stitches would normally be needed or as a first step before stitches. The current from the device can be adjusted remotely through a cell phone or other connection by hospital personal or paramedics as well as information sent on vitals through a port created for that connected to a pad on the patient. Programs can be created and modified for different types of injury.

### DESCRIPTION OF THE DRAWINGS

FIG. 1 presents a view of a body with a pad placed over the heart area

FIG. 1a presents a view of a body with 2 pads placed side by side over the heart area

FIG. 1b presents a view of a body with 2 pads placed top and bottom over the heart area

FIG. 2 presents a view of a body with pads placed over the lungs

FIG. 3 presents a view of a body with pads placed over the heart and lung areas.

FIG. 4 presents a view of 3 pads placed in a configuration over the heart area.

FIG. 5 presents a view of 3 pads placed in a configuration over the heart area along with 2 pads placed on the sides.

FIG. 6 presents a view of a large pad placed covering heart and lung areas.

FIG. 7 presents a view of a band with a pad placed covering the heart and lung areas.

FIG. 8 presents a view of a pad placed over the abdomen.

FIG. 8a presents a view of 4 pads placed over the abdomen.

FIG. 9 presents a view of 4 pads placed covering the pressure points with major arteries.

FIG. 10 presents a view of a strap around an appendage.

FIG. 11 presents a view a pad placed on an area of the body.

FIG. 12 presents a view of a controlling device that can be adjusted to different settings.

FIG. 13 presents a view of a pad with a contact point.

FIG. 14 presents a view of a contact point at the end of a connecting wire.

FIG. 15 presents a view of a single pad with 3 connection points

FIG. 16 presents a view a single pad with one connection point.

FIG. 17 presents a view of a directing and reflecting pad.

FIG. 18 presents a view of a controlling device with single wires from 2 ports to 2 pads.

FIG. 19 presents a view of a controlling device with a single wire split to cover 2 pads.

FIG. 20 presents a slow chart for using a program on a device and using a device manually.

FIG. 21 presents a flow chart for transitioning a patient from Electronic CPR to their heart beating on its own.

### ITEMS IN FIGURE DRAWINGS

1. Patient
2. Pad or connection device placed over heart
3. Pad or connection device placed over lungs
4. Large pad or connection device placed over heart and lungs
5. Four pads or connection devices placed over abdomen or over four pressure points on main arteries for limbs
6. Large pad or connection device placed over abdomen
7. Strap connection device placed around limb
8. Pad or connection device placed over wound on limb
9. Pad or connection device
10. Electronic device like a TENS or EMS unit that can be programmed, used manually and modified
11. Screen on electronic unit
12. Adjustment buttons on electronic unit to choose and adjust program
13. Wire connecting electronic device to pad or connection device
14. Connector on pad or connection device to connect wire to
15. Connection point on pad or connection device that wire connects to
16. Connection point on wire that connects into pad or connecting device
17. Outer support area of connection point on wire that connects into pad or connecting device
18. Pad or connection device placed on side of lungs
19. Band placed over chest area covering heart and lungs
20. Knob or dial that can manually be adjusted to adjust intensity of the electrical flow on electronic device.
21. On and off switch that can be used to turn electronic device on and off and also send pulses of electricity manually.

### DETAILED DESCRIPTION OF THE INVENTION

The invention generally relates to controlling the heartbeat, blood flow and bleeding. In particular, the invention provides a way to use the bodies own muscles and tissue to control blood flow using a portable, small-footprint electrical device like a TENS unit connected to the body using wires connected to pads/connection points placed on a persons body. The body is controlled by electricity. We use the electric unit to send electricity to the body to over rule the bodies signals and control the body with our electrical input. During cardiac arrest we send electricity to the heart in pulses to tell it to compress and release which pumps blood. This can be done with an automatic program or manually. The intensity of the electricity can be controlled manually and adjusted for the patients size, weight and other factors. To control bleeding we send electricity to the body to constrict the muscles and blood vessels, electricity flows through the path of least resistance best, to slow or stop bleeding which aids in coagulation and helps prevent shock and infection. As the healing process proceeds the amount of blood flow can be adjusted. This medical procedure works on both internal and external injuries to control bleeding.

Controlling a heartbeat using an external electronic device to override the bodies electronic signals to the heart. 1. Connect wires to electronic device on one end and pads or connection device on the other end, 2. place pads on subject over heart area, 3. turn on device and turn control up to increase current evenly for about ¾ to 1 second and then back to zero, 4. continue this pattern to compress the heart and then release it, compress and release, to pump blood to the body, keep heartbeat until medical help arrives and can use a defibrillator.

If patient heart has flat lined or is not responsive use steps above to create a heartbeat and reintroduce electricity into the heart and then try defibrillator.

Transitioning from electronic device control to heart on its own. 1. Stop using device and see if the heart continues on a regular beat, 2. Alternative set the device to send current and press button to turn the device on and off quickly at one second intervals to act as an external pacemaker.

This medical procedure steps in the method are 1. attach wires to electronic device like a TENS unit, 2. attach wires to pads or connection devices, 3. place pads on patient, 4. turn on electrical device, 5. increase intensity of electrical current from zero to desired intensity over a ¾ to 1 second time and then decrease intensity to zero for ½ to 1 second, 6. Repeat step 5 multiple times either manually or using a preprogrammed setting on the electronic device, 7. When patients is responding cease current to see if their heart begins beating on its own, if not sent quick bursts of electricity to the heart in intervals like a pacemaker, if this does not work resume step 5 until medical help arrives to keep the heart and brain in their best condition for defibrillation or what ever method the medical professionals deem best.

Check for heartbeat for 10 seconds in step 7 before sending pacemaker type bursts to try to restart the heart for 10 seconds and immediately resume step 5 if no pulse is found.

Defibrillation shocks the heart to stop the fast pace and mixed electrical signals of the heart by overpowering the hearts signal and the persons heart must restart again on its own.

We choose to send an electronic current to the heart that squeezes the heart instead of shocking it. The electric current we send also overpowers the signals from the heart but in a gentler way that actually produces a heartbeat and introduces electricity into the heart at the same time. This resets the heart to a regular pace that it can then take on its own or if needed it can be shocked to stop and restart but we have kept it healthy enough to do so.

Set the device to send current and press button to turn the device on and off quickly at one second intervals to act as an external pacemaker.

The electronic device is a TENS or EMS type unit but not limited to and modifications can be made to it as well.

Program an automated program into the electronic unit to set the rhythm and initial intensity of the current. Program can be to form a heartbeat by increasing the current from zero to a set intensity and then back to zero for an interval to release the heart and let blood back into it before compressing the heart again to pump that blood back out.

Program the electronic unit to send an electronic pulse like a pacemaker at a set interval.

Create an adjustable intensity setting and dial on electronic unit to adjust for patient size, weight and other health considerations.

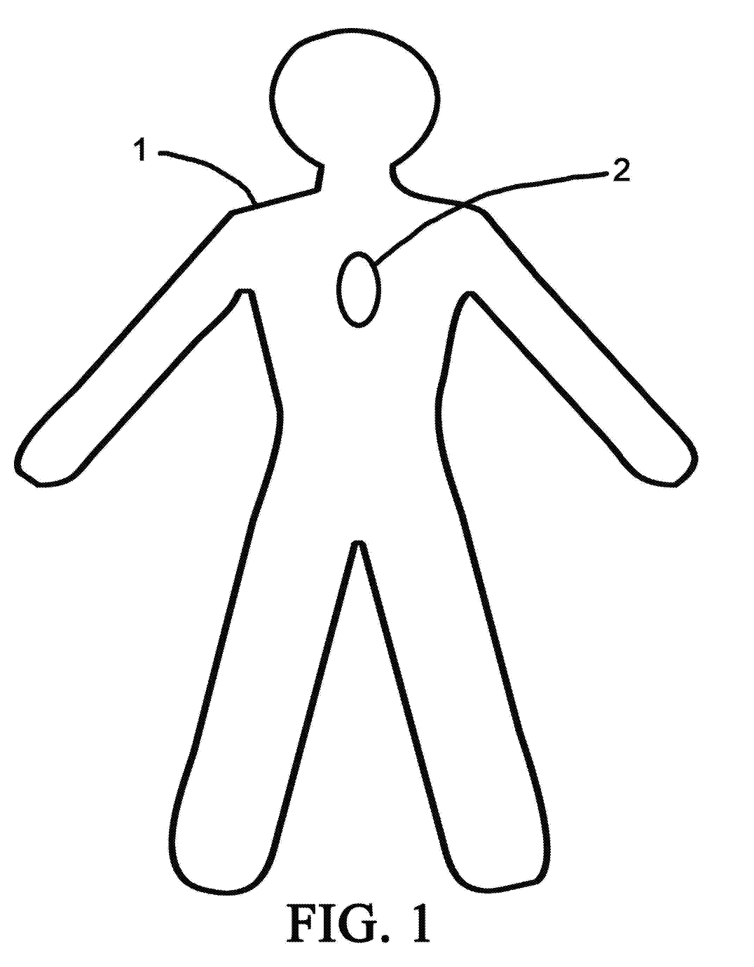

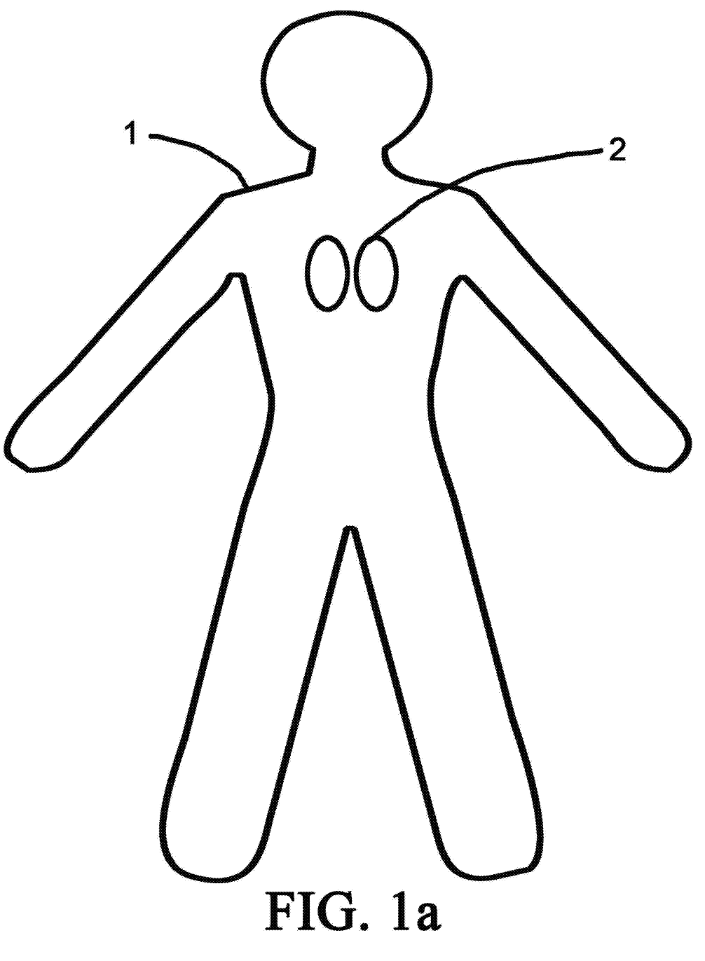

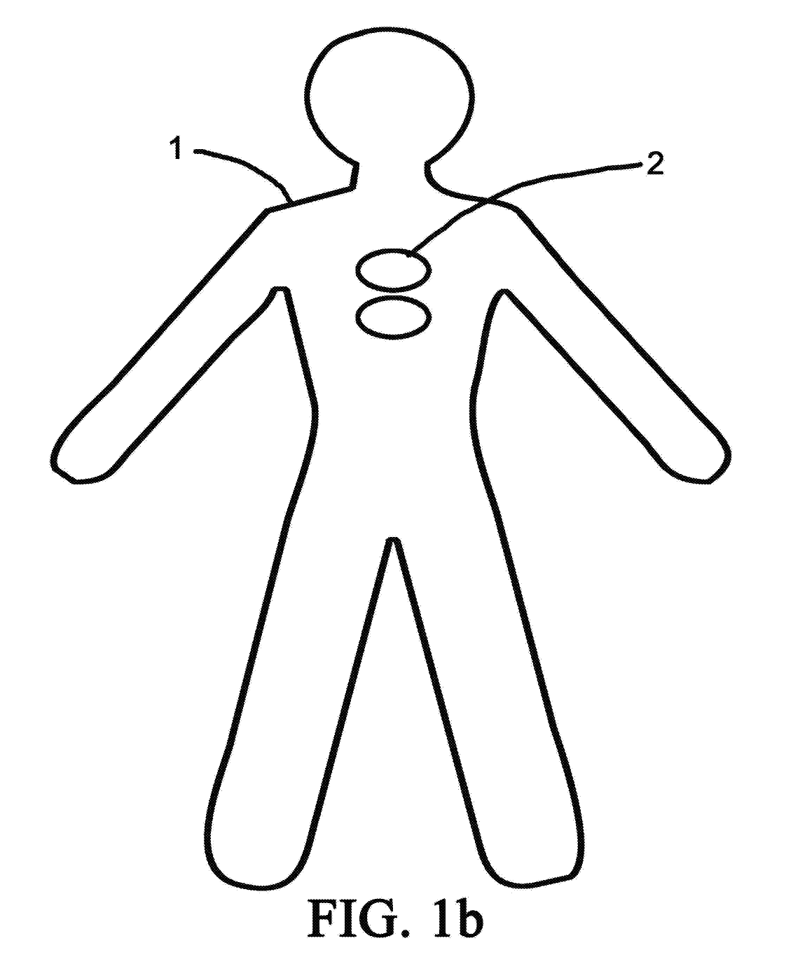

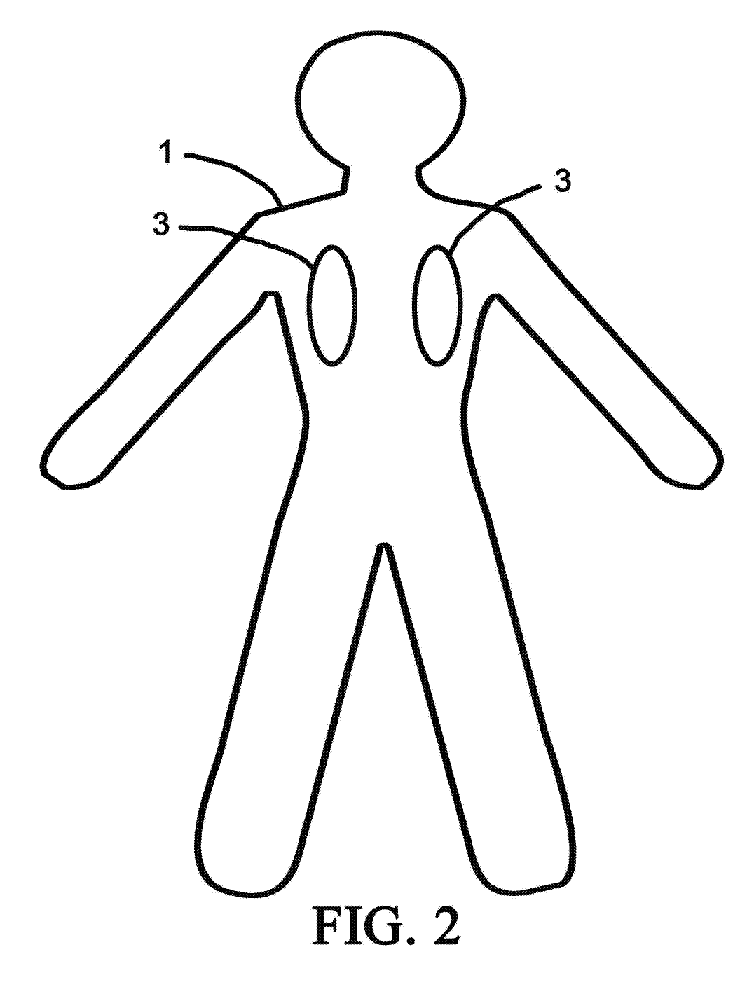

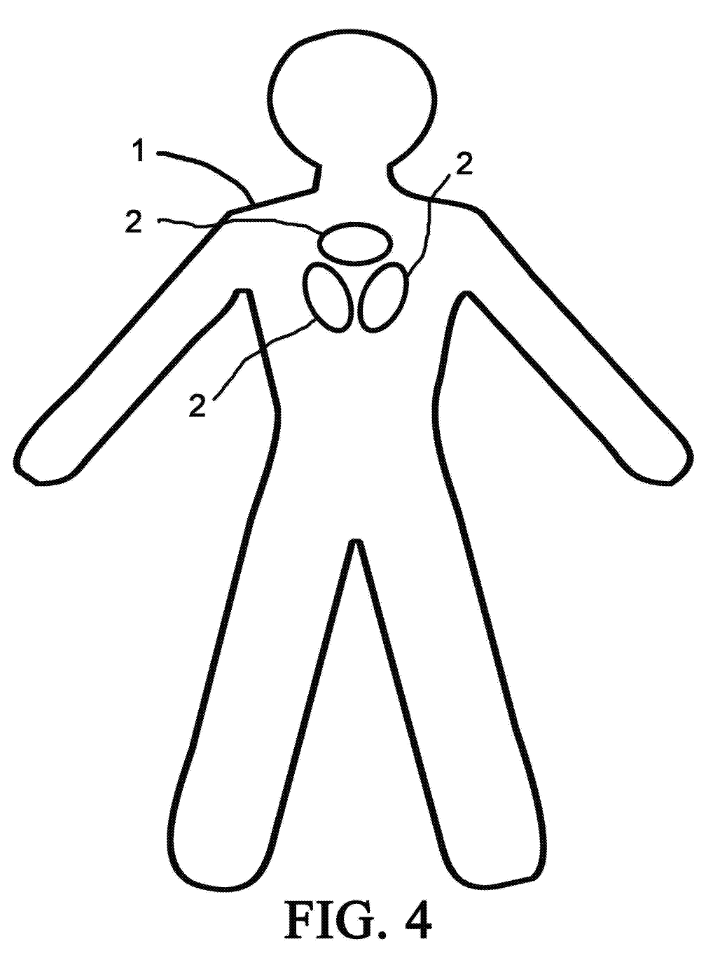

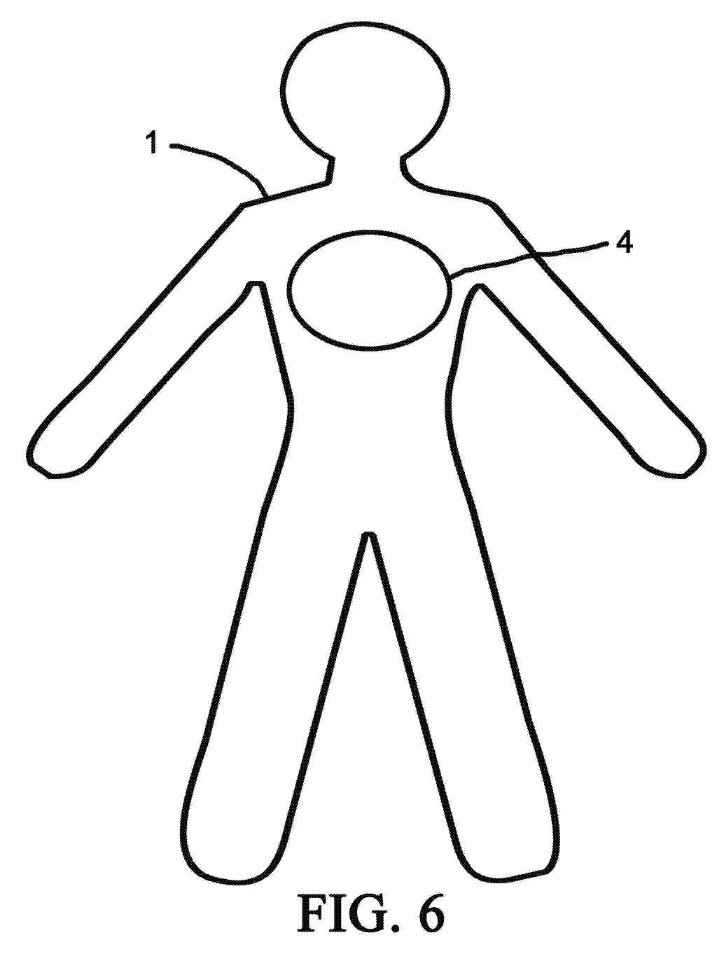

Place a pad over the heart and under the heart on the back FIG. 1, place a high and low pad FIG. 1b or a left and right over the heart FIG. 1a, 2. Place a large pad over the heart area FIG. 6, 3. Place 3 pads on heart area FIG. 4

Send electricity to the left and then the right pad FIG. 1a, to compress the left and then right side of the heart before stopping interval to release the heart. Use this technique with the top and bottom, diagonal, or any heart pad placement.

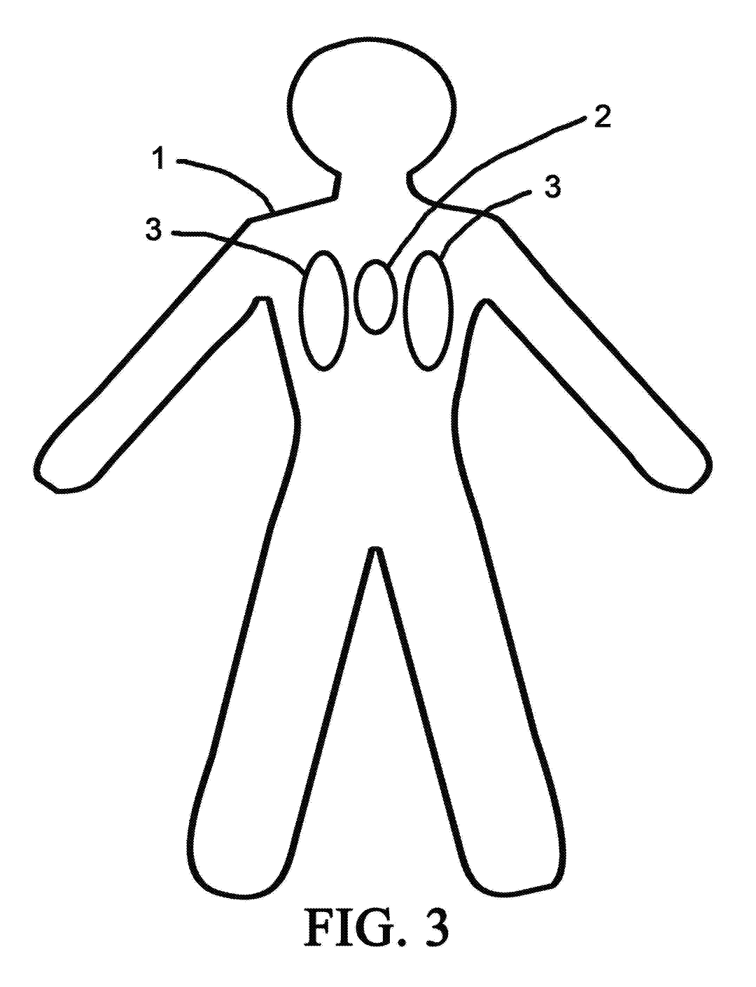

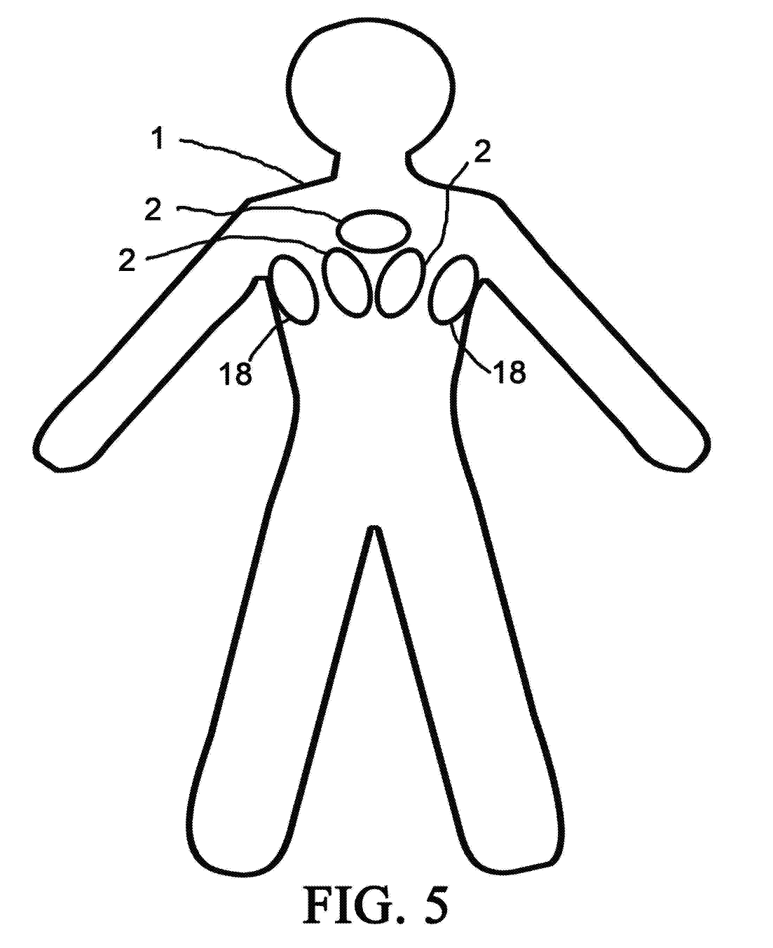

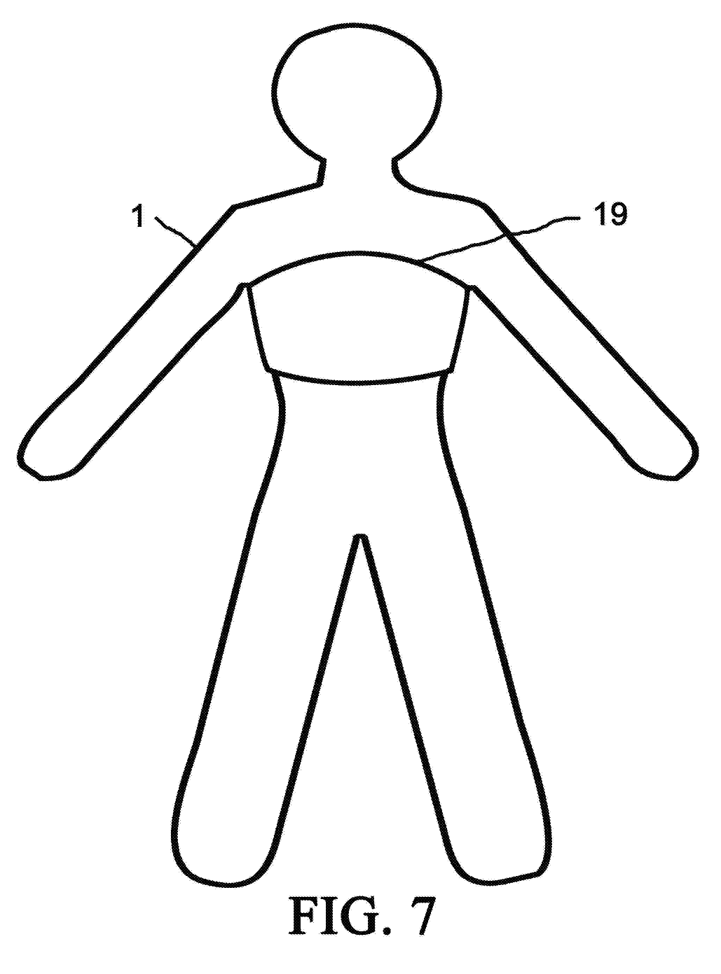

To increase breathing place separate pads on lung area FIG. 3 and control them separately or by using a second unit to pace them slower than the heartbeat. Alternative, place 3 pads over heart and two on lungs at sides FIG. 5, Place a band around the chest FIG. 7

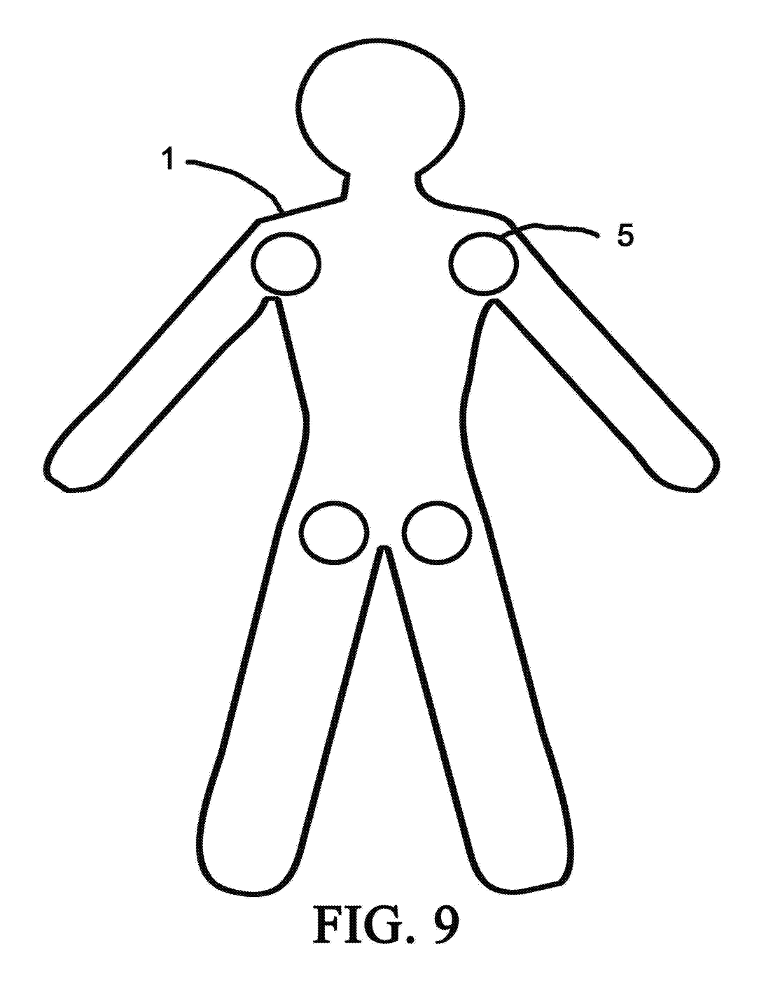

To increase blood flow to the heart and brain place a large pad over the abdomen FIG. 9, or place 4 pads over the abdomen and send a steady current to constrict the abdomen area restricting blood flow to that area.

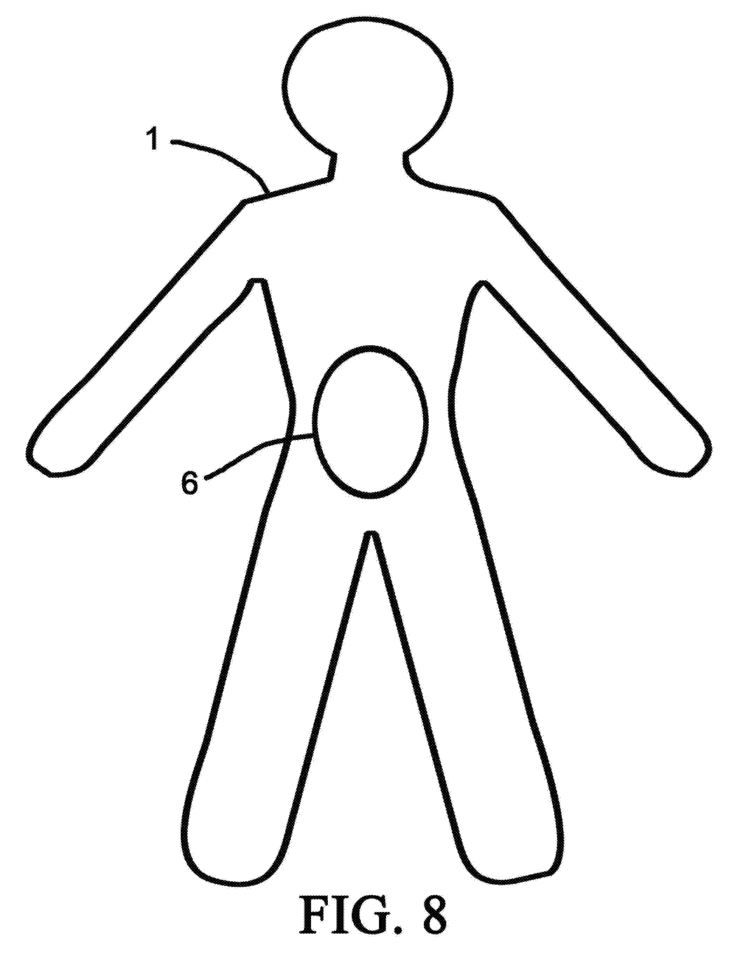

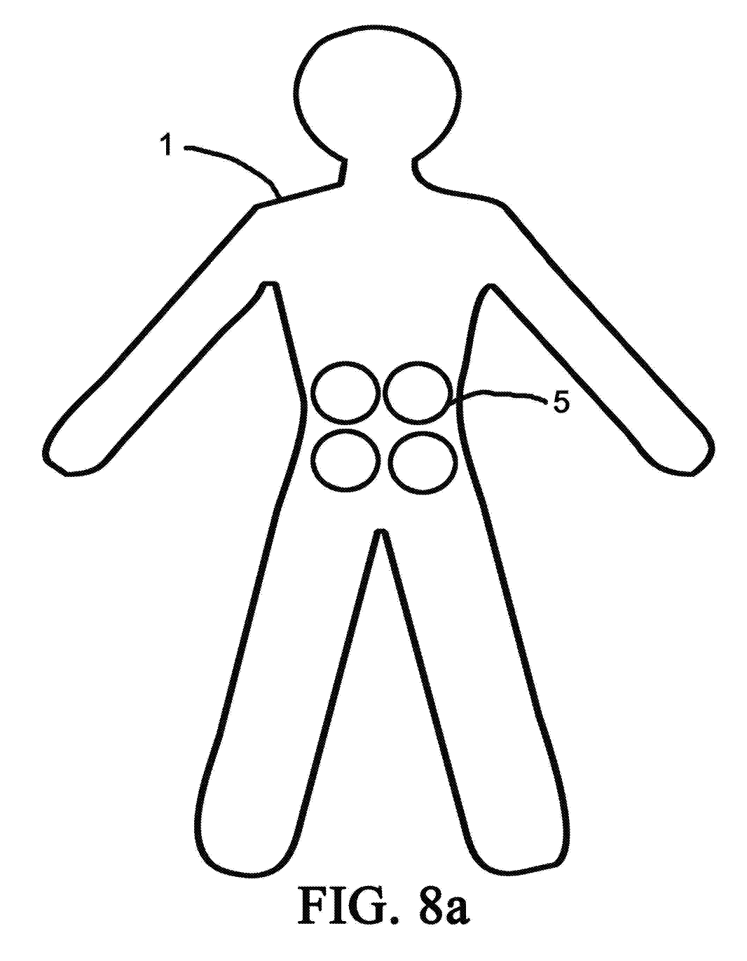

Place pads on the pressure points for the arms and legs FIG. 8a to constrict main arteries and directing a steady current to them to restrict blood flow to the limbs and increase blood flow to the brain and heart.

Place pads on a pressure point for an arm or leg FIG. 8a to constrict the main artery and direct a steady current to the pads to restrict blood flow to the limb to reduce bleeding from an injury to that limb. Use this in conjunction with a tourniquet to require less pressure.

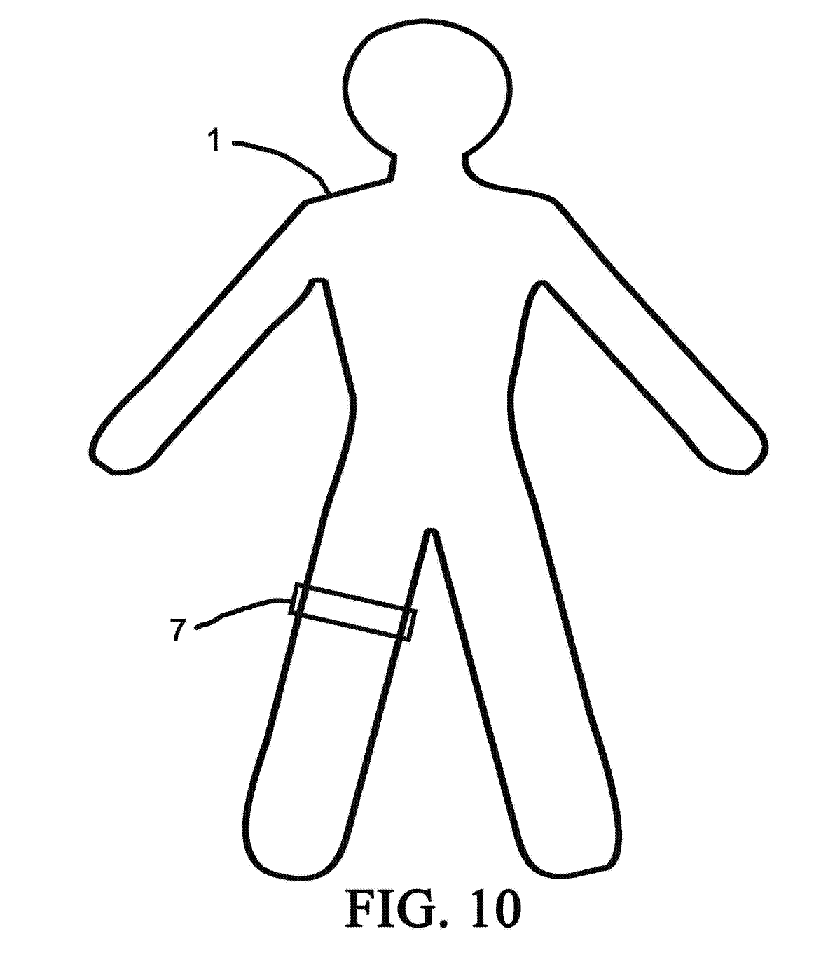

Place a series of pads or a connecting strap FIG. 10 around an arm or leg 3 inches above a wound and send a steady current to restrict blood flow.

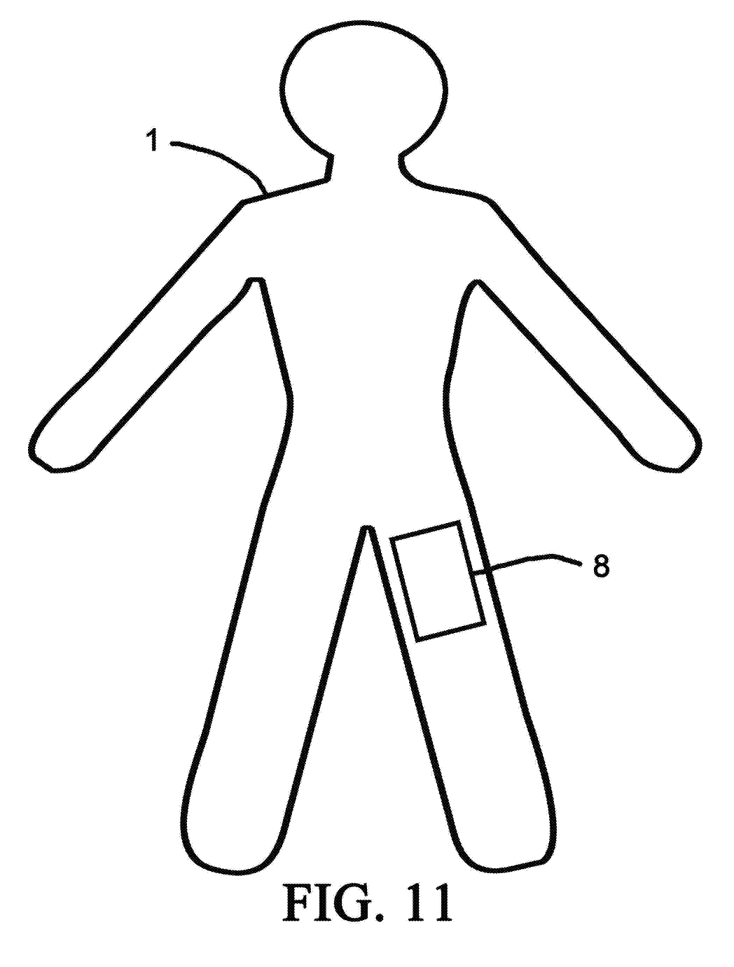

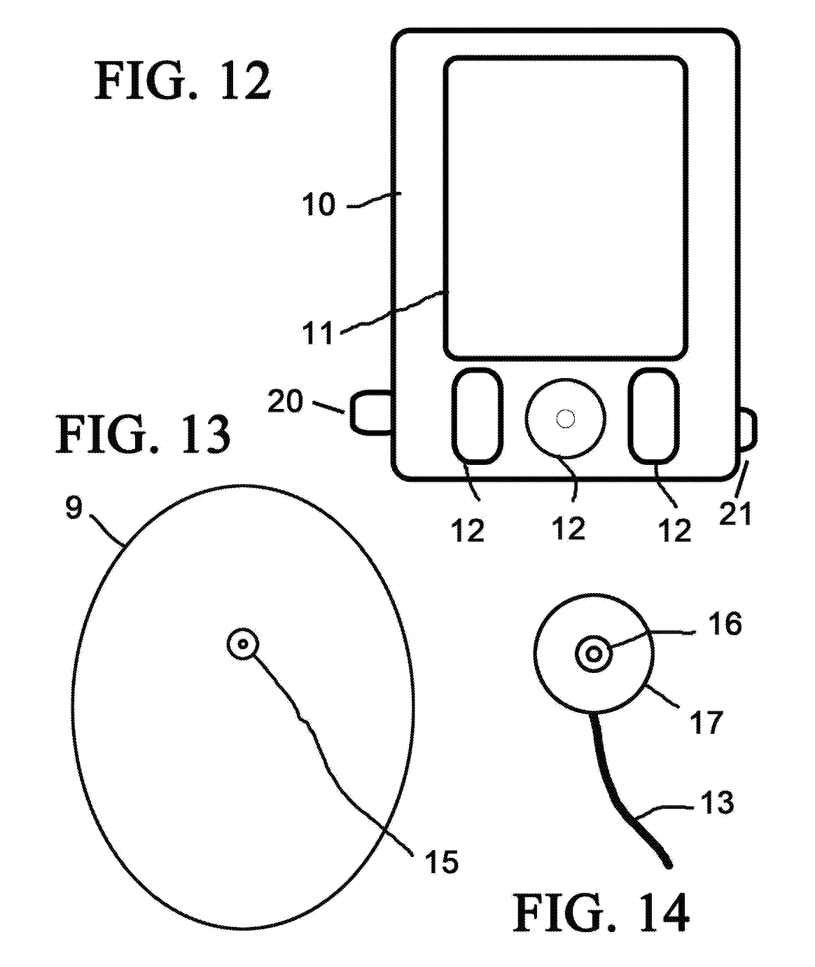

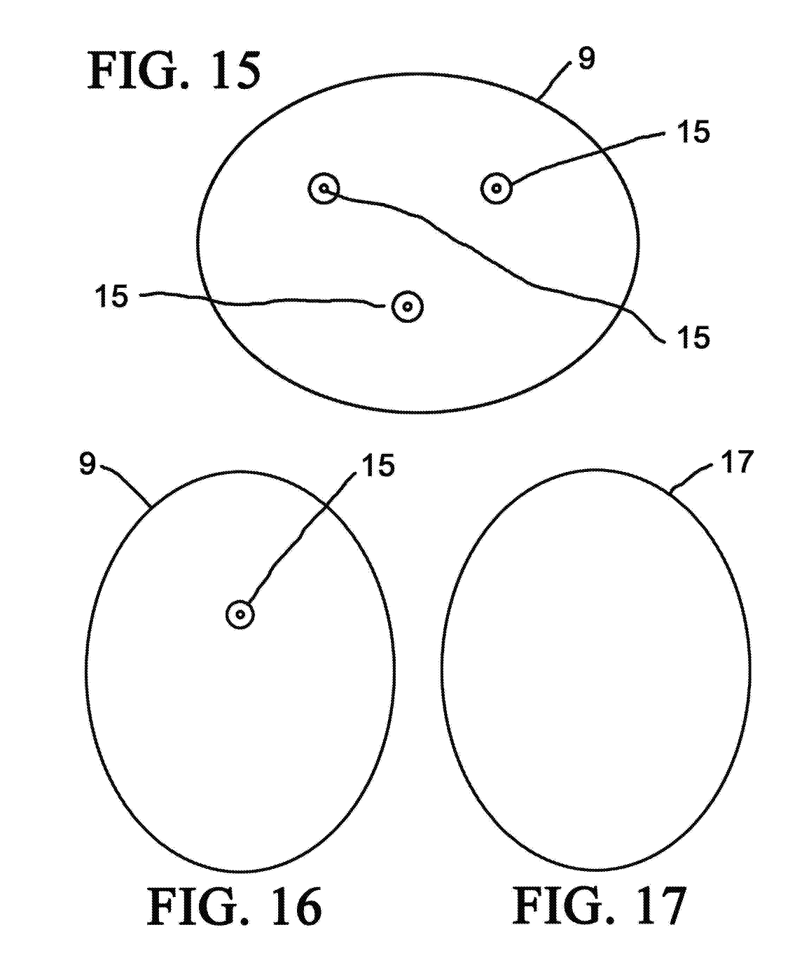

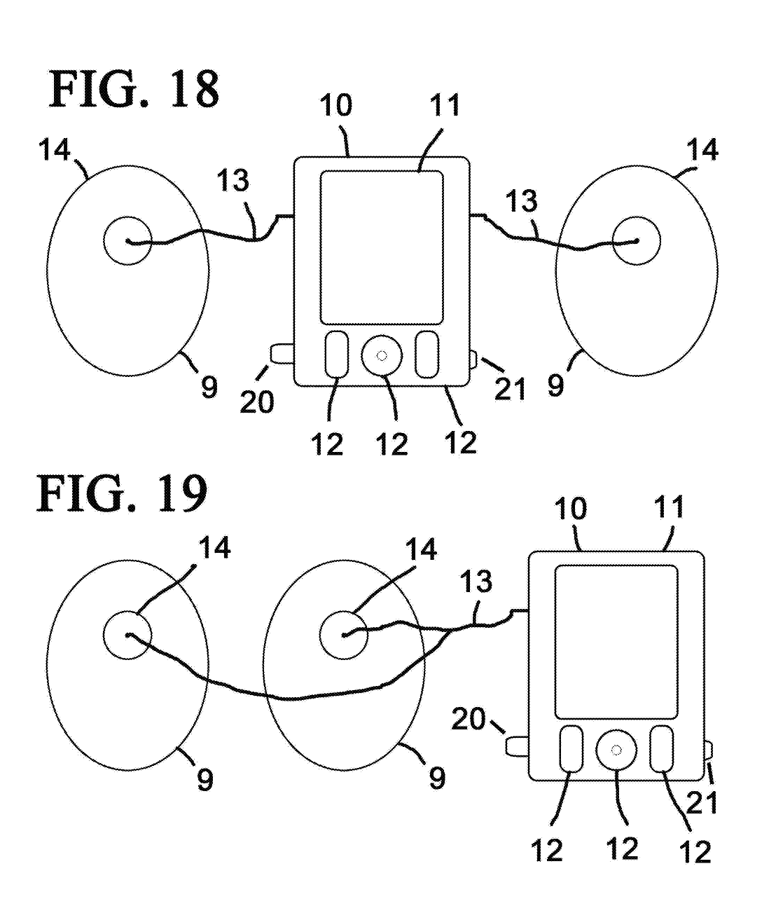

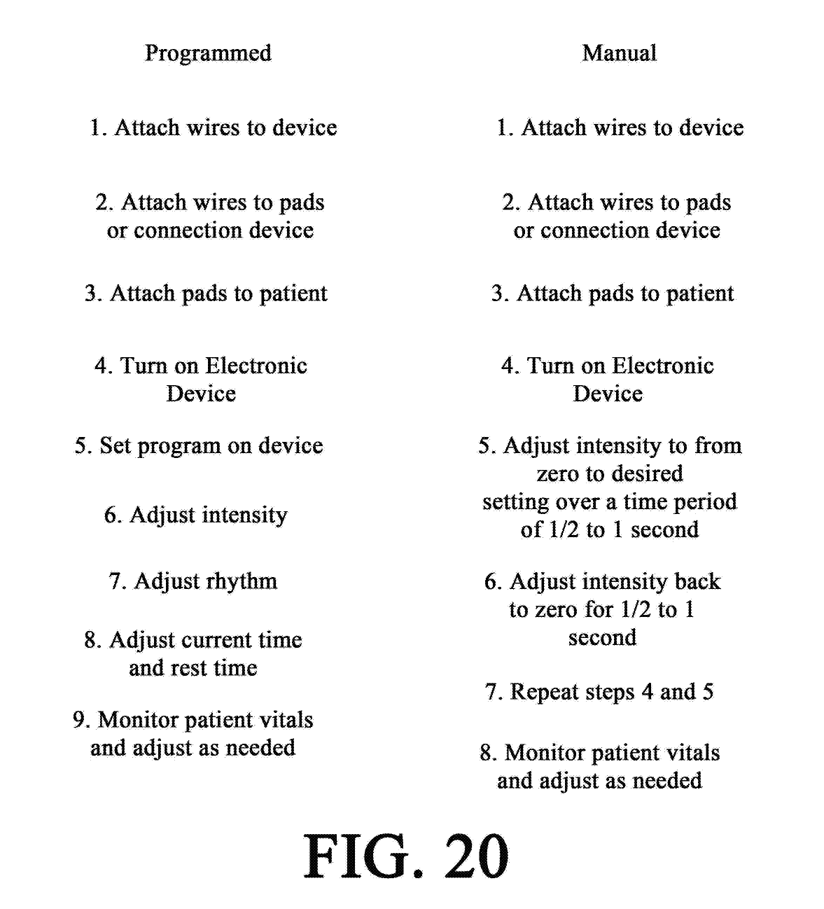

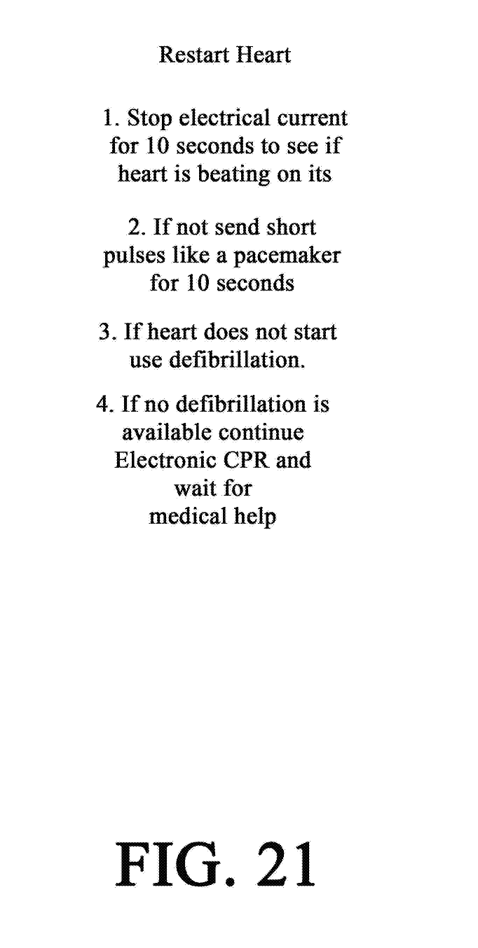

Place a pad over a wound FIG. 11. Send a steady electrical current to the pad to constrict the area and blood vessels to control bleeding. Slowing down bleeding will increase coagulation, and lessen the risk of shock and infection.

Use a clear pad to be able to view how the wound is healing.

Velcro straps or other ways of placing a pad on a wound may be needed if it is bleeding heavily.

For bullet and knife or puncture wounds place a pad over the wound area and increase the intensity of the electrical flow to reach the depth of the wound inside the body and restrict bleeding.

For bullet wounds place a wire or small thin connection device into the wound to constrict the area around the wound and reduce blood flow.

For a deep knife wound place a pad or connection device inside the wound to constrict blood inside the wound.

Cut a large pad to fit the wound area as needed.

Place a pad or series of pads over an internal injury area and send a steady current of electricity to constrict the area and control bleeding.

Periodic release of the current to allow some blood through as is needed to for healing.

Place a pad the size needed over a burn and send a steady current of electricity to control bleeding and damage immediately after a burn. This is an alternative to cold water if there is none around.

Place pads around an incision during surgery and send a steady current through the pads to control bleeding. Adjust the intensity of the flow as needed.

Ceramic scalpels and instruments as well as insulation will allow electricity in the area.

Place a pad or connection device inside the area being operated on and send an electronic current to reduce bleeding.

Place a pad on the skin over or near the area being worked on and send an electronic current to restrict blood flow.

Send an electronic flow through a clamp to reduce blood flow.

Less blood flow means less suction and less blood loss.

Place a pad over an incision to help seal them instead of or with stitches.

Keep a steady flow of electricity to an area to reduce bleeding.

Let some blood through to an area in the amount you want to aid in healing for the amount of time needed.

Connect electronic device to a cell phone to let it be adjusted remotely from a hospital or medical person.

Connect a pad to a port on the electronic device to take vital signs of a patient to help the hospital know how to adjust the electrical flow.

Use a second device to send vital signs of a patient to help the hospital know how to adjust the electrical flow or current.

Record a persons natural heartbeat and program that into the device.

Adjust how the automatic setting is programmed to build to a peak and how it fades.

Create a cell phone app using an exterior battery for additional power.

Place a magnet on the other side of the arm, leg, body to direct and focus the flow of electricity.

Place a reflecting pad made of metal or other material to focus the electricity to an area.

Build port into electronic device to let you connect a battery for longer battery life.

Construct a pad to diffuse electricity for a broader area, an area that affects the surface more.

Construct a pad to focus electricity to target a specific area and be able to go deeper into the body.

Create automatic programs to work with stents, pacemakers and heart conditions.

Create settings to be used on a person who has metal like a stent or pacemaker in their heart.

Check medic alert bracelet for pacemaker, stent and other medical reasons to use or not use electric current.

Set electronic device for a low enough setting with longer intervals that allow cooling to use on a person with stents, a pacemaker, valve or other object in their heart area.

Electronic bleeding control procedure method, 1. Connect wire to electronic device like a TENS or EMS unit, 2. Connect other ends of wire to a pad or connection to patient device, 3. Place pad(s) on patient where needed and most effective, 4. Turn on unit and set intensity to control the blood flow (manual or automatic settings can be set), 5. Monitor patient and area that is bleeding and adjust unit as needed with the passing of time and as the healing process continues for optimum results.

Use a clear pad or connection device that allows you to see the wound area to monitor how the healing process is going and adjust the electronic device accordingly.

Use a strap or connecting device to hold a pad on a wound if it is bleeding heavily and won't take the standard connection gel or other surface connectors.

Use a pad with a sticky surface to help attach them to the patient and increase conductivity.

Advantages of these medical procedures include:

1) Defibrillation shocks the heart to stop it and hopes it will start again on its own, it does not work with many heart situations. Electronic CPR compresses and releases the heart by overriding the bodies electrical signals with and electronic device sending signals we can control that keeps the heart beating no matter its state, keeps it in a better state for defibrillation if it is used and can bring it back into a regular heartbeat on its own.

2) CPR must be learned, our Electronic CPR can be applied using a program.

3) CPR cannot be self administered, Electronic CPR can be applied by the patient themselves.

4) CPR is physically exhausting and can only be done for 4 to 5 minutes by one person at a time which is a problem if only one person is there, if response time is longer than 5 minutes and getting the patient to a medical facility takes longer than 5 minutes. Electronic CPR can last as long as the batter in the electronic device lasts and external batteries and power sources can be used to keep it going as long as needed.

5) CPR causes bruising, lacerations and other injuries. Electronic CPR uses the bodies own muscles to keep the heart beating and blood flowing.

6) Defibrillation requires an expensive unit which may be 5 minutes away if one is available. The first 5 to 10 minutes are critical in a heart attack and the brain may be dead and heart tissue dying by the time they get the defibrillator. Electronic CPR devices are small, smaller than a cell phone, and inexpensive so they can be placed in more places economically and carried easily on a patient or member of the public. Kind of like carrying your own jumper cables for your body. This makes them quicker to use when minutes count. Getting Electronic CPR started can take less than 30 seconds.

7) CPR is very inefficient even when done by a professional it only makes 20 to 30% of the blood flow from a normal heartbeat. Done by an inexperienced bystander it can be even lower.

8) Electronic CPR can be adjusted remotely by connecting the device to a cell phone either wired or wirelessly so a professional can adjust the settings for the patients needs. Vital signs can also be sent to the hospital or medical facility to help them set the settings for the patient. Vital signs can be measured with the electronic device through a separate port and pad or by using a second device.

9) A basic electronic unit with only manual controls is very inexpensive to produce for use in poorer countries and makes it more affordable to keep on in your car, office, home etc.

10) Sending a steady current to the abdomen and main arteries in the arms and legs to restrict blood flow to those areas increases blood flow to the brain and heart during a heart attack or heart episode.

11) Over weight people are more likely to get a heart attack and can be harder to administer CPR. With Electronic CPR it is set on the heart area and does not have to compress the whole chest and ribs. The intensity of the electrical flow/current can be increased if needed and adjusted for the patient.

12) Tourniquets are generally a measure of last resort. Life or limb is the choice, you will lose one or the other. Using an electronic device you reduce blood flow by restricting the pressure point or main artery for the limb overall by placing a pad over it and sending a steady flow of electricity to the area.

13) Using the electronic device we have discussed you can attach a wire to a strap to place around the limb and distribute the electrical current to restrict the blood flow but be able to fine tune it and even let some blood flow through at intervals and different levels to help keep the limb healthy.

14) Placing a pad that can be cut to the size needed for a wound over the wound and sending a steady stream of electrical current through it from an electronic device with constrict the muscle and tissue to constrict the blood vessels and reduce bleeding.

15) Reducing bleeding with electronic bleeding control helps prevent shock, infection and aids in coagulation.

16) In surgery blood loss reduce bleeding using electronic bleeding control.

17) Use ceramic scalpels and insulated instruments when using electronic bleeding control.

18) Suction is needed less when bleeding is controlled using electronic bleeding control.

19) Cauterizing, clamping and tying off damage tissue and create scar tissue. Use electronic bleeding control for less tissue damage.

20) Internal bleeding is hard to stop. Use electronic bleeding control to stop internal bleeding and aid in healing.

21) Putting cold water on a burn immediately reduces damage to the skin and underlying tissue. If you don't have cold water place a pad on the affected area, it may need to be a large pad in some cases, and send a steady electric current to constrict the skin and tissue will help reduce damage.

22) Change pad placement and size according to your patients size, weight, age, medical history, and injury being treated.

23) Connect wires to the best connection device for your patient, their injury and situation. Pads and connection devices can be made of different materials and be different shapes and sizes.

24) Use the electronic device to stimulate an organ or muscles with an electrical current to constrict and release the organ or area using the steps used for Electronic CPR.

We claim:

1. A method for exerting a compressive force on the heart and then releasing it to create a heartbeat that pumps blood, comprising the steps of 1) attach wires to electronic device like a TENS or EMS unit, 2) attach wires to pads or connection devices, 3) place pads on patient, 4) turn on electrical device, 5) increase intensity of electrical current from zero to desired intensity needed to compress the heart and then decrease intensity to zero, 6) repeat step 5 multiple times to keep the heart beating and blood flowing as long as is needed.

2. The method of claim 1, wherein the time of increasing the electrical current is increased gradually to smoothly compress the heart and pump the blood up to 4 seconds and the release time where no electrical current is sent to the body is up to 10 seconds adjusting according to patient results with ¾ of a second compression and ½ second release being the preferred embodiment but not limited to.

3. The method of claim 1, wherein the intensity of the electrical current is adjusted for the size, weight, age and medical condition of the patient and the results of the heart.

4. The method of claim 1, wherein the electrical current is controlled by adjusting the electronic device manually or using a preprogrammed setting on the electronic device which can be adjusted as well with multiple programs available.

5. The method of claim 1, wherein the placement of the pads on the left or right, top or bottom of the heart, heart and back, are preferred embodiments but not limited to.

6. The method of claim 1, wherein the electronic device is connected to a cell phone or other transmitting device using wires or wirelessly to allow adjustment of the device from a remote location.

7. The method of claim 1, wherein the electricity from the unit is stopped for a short period to see if the patients heart has resumed beating on its own.

8. The method of claim 1, wherein the electricity from the unit is changed from a compress and release cycle to a short burst of electricity like a pacemaker, alternative set the device to send current and press button to turn the device on and off quickly at one second intervals to act as an external pacemaker, program the electronic unit to send an electronic pulse like a pacemaker at a set interval.

9. The method of claim 1, wherein the method is used with a flat lined patient or other heart condition or state where defibrillation will not work to create a heartbeat, create blood flow and add electricity to the heart.

10. The method of claim 1, wherein the patient sets up the device and uses it on themselves.

11. A method for exerting a compressive force on the body to constrict blood vessels and reduce blood flow to an area, comprising the steps of 1) attach wires to electronic device like a TENS or EMS unit, 2) attach wires to pads or connection devices, 3) place pads on patient, 4) turn on electrical device, 5) increase intensity to the desired level to control the blood flow and bleeding.

12. The method of claim 11, wherein the amount of electrical current and restriction of blood flow is adjusted with the healing process and over time.

13. The method of claim 11, wherein a pad, pads or connection device is place on the abdomen and a steady electrical current is sent to compress the abdomen and constrict blood flow to increase blood flow to the brain and heart.

14. The method of claim 11, wherein a clear pad is used to see how the healing process is going on a wound.

15. The method of claim 11, wherein a pad, pads or connection device is placed over an internal injury or organ to constrict blood vessels and control bleeding by sending a steady electrical current to that area.

16. The method of claim 11, wherein electronic bleeding control is used in surgery to control bleeding at the incision and in the area being operated on being delivered through pads or other connection devices including forceps, clamps and other surgical tools.

17. The method of claim 11, wherein the electrical device is connected to a strap or group of pads placed above the wound on a limb to restrict blood flow like a tourniquet but allowing enough blood to go through to help keep the limb healthy.

18. The method of claim 11, wherein the pad is placed over a burn area and a steady current is sent to constrict the skin and tissue in the burn area to reduce damage to the burn area.

19. The method of claim 11, wherein the pad is place above a bullet or knife wound or a connecting device is placed inside the wound to constrict the area around it to close the wound.

20. The method of claim 11, wherein the wire from the device has a tip on it that can be inserted into a major artery to control bleeding when electricity is sent through the wire in a steady flow.
